(19) United States
(12) Patent Application Publication
(10) Pub. No.: US 2023/0207083 A1
(43) Pub. Date: Jun. 29, 2023

(54) METHODS AND SYSTEMS FOR INDIVIDUALIZED RISK SCORE ANALYSIS

(71) Applicant: KONINKLIJKE PHILIPS N.V., EINDHOVEN (NL)

(72) Inventors: Claire Yunzhu Zhao, Cambridge, MA (US); Kristen Tgavalekos, Cambridge, MA (US); Shreyas Raj Ravindranath, Cambridge, MA (US)

(21) Appl. No.: 17/926,816

(22) PCT Filed: May 26, 2021
(86) PCT No.: PCT/EP2021/064111

§ 371 Date: Nov. 21, 2022

Related U.S. Application Data
(60) Provisional application No. 63/030,442, filed on May 27, 2020.

(51) Int. Cl. G16H 15/00; G16H 50/30; G16H 50/20; G16H 50/70

(52) U.S. Cl. CPC G16H 15/00; G16H 50/20; G16H 50/30; G16H 50/70

(57) ABSTRACT

A method (100) for generating and presenting a patient risk score, comprising: (i) receiving (104) a plurality of features about the patient comprising at least a plurality of vital signs obtained for the patient; (ii) characterizing (106), using a trained risk model, an importance of each of the received plurality of features to a risk score analysis; (iii) calculating (108) an initial risk score; (iv) identifying (110) one or more missing features; (v) calculating (110) a risk score confidence interval comprising an effect of the identified one or more missing features on a confidence range of the initial risk score; (vi) calculating (112), from the initial risk score and the calculated risk score confidence interval, a risk score range; and (vii) presenting (118) the risk score range comprising the initial the score plus and minus the calculated risk score confidence interval.

### FIELD OF THE DISCLOSURE

The present disclosure is directed generally to methods and systems for generating and presenting improved patient risk scores with confidence levels using a risk score analysis system.

### BACKGROUND

Clinical decision support tools are designed to provide targeted and relevant information healthcare providers at key times during care. These tools guide medical diagnosis and inform therapeutic decisions, and have been shown to improve the performance of healthcare providers. Many current clinical decision support tools comprise machine learning (ML)-based clinical support systems. These ML-based tools have been shown to out-perform rule-based systems in predicting patient outcomes.

Some ML-based tools comprise patient-specific risk scores, indicating a risk of an outcome in view of one or more input features. The interpretation of machine-learning risk scores is vital for their ability to support clinical decisions and transitions of care, and thus quantifying the level of certainty in a risk score prediction can reduce false alarm rates and further encourage clinicians’ interpretation.

Many ML-based tools comprising risk scores will either output a risk score regardless of certainty or not output score at all. Alternative methods exist to quantify uncertainty within a machine-learning model, but they focus on delineating model (epistemic) uncertainty and data (aleatoric) uncertainty, and do not relate to feature importance interpretation.

In contrast, ML-based tools that also communicate certainty or uncertainty, and also relate uncertainty measures to feature importance and contribution to the output score, provide clinical context and thus have the potential to enhance incorporation of risk scores in the clinical workflow to aid medical decisions by identifying patients at risk for deterioration and determining appropriate levels of care.

### SUMMARY OF THE DISCLOSURE

There is a continued need for ML-based clinical support methods and systems that quantify and communicate uncertainty within a machine-learning patient risk score model. Various embodiments and implementations herein are directed to a method and system configured to generate and present a patient risk score using a risk score analysis system. The system receives a plurality of features about the patient, where the plurality of features comprise a plurality of vital signs obtained for the patient at a first time point. The system characterizes, using a trained risk model of the risk score analysis system, an importance of each of the received plurality of features at the first time point to a risk score analysis. The system calculates, from the received a plurality of features about the patient, an initial risk score. Then the system identifies, using the trained risk model, one or more missing features each comprising a feature not found in the plurality of received features, wherein each of the one or more missing features is relevant to the patient risk score calculation. The system calculates, using the trained risk model and the identified one or more missing features, a risk score confidence interval comprising an effect of the identified one or more missing features on a confidence range of the initial risk score, and then calculates, from the initial risk score and the calculated risk score confidence interval, a risk score range. The system prevents, to a user via a user interface of the risk score analysis system, the risk score range comprising the initial the score plus and minus the calculated risk score confidence interval, and one or more of the identified one or more missing features.

Generally, in one aspect, a method for generating and presenting a patient risk score using risk score analysis system is provided. The method includes: (i) receiving, at the risk score analysis system, a plurality of features about the patient, the plurality of features comprising at least a plurality of vital signs obtained for the patient at a first time point; (ii) characterizing, using a trained risk model of the risk score analysis system, an importance of each of the received plurality of features at the first time point to a risk score analysis; (iii) calculating, from the received plurality of features about the patient, an initial risk score; (iv) identifying, using the trained risk model, one or more missing features each comprising a feature not found in the plurality of received features, wherein each of the one or more missing features is relevant to the patient risk score calculation; (v) calculating, using the trained risk model and the identified one or more missing features, a risk score confidence interval comprising an effect of the identified one or more missing features on a confidence range of the initial risk score; (vi) calculating, from the initial risk score and the calculated risk score confidence interval, a risk score range; and (vii) presenting, to a user via a user interface of the risk score analysis system, the risk score range comprising the initial the score plus and minus the calculated risk score confidence interval, and one or more of the identified one or more missing features.

According to an embodiment the method further includes: comparing the risk score range to a predetermined risk score threshold; determining, by the trained risk model, the risk score to be certain if the risk score range is outside the predetermined risk score threshold, or determining the risk score to be uncertain if the risk score range is within the predetermined risk score threshold; and presenting, to the user via the user interface, the determination of the risk score to be certain or uncertain.

According to an embodiment, the risk score is determined to be certain if the risk score has been stable for a predetermined period of time, even if the risk score range is within the predetermined risk score threshold.

According to an embodiment, the risk score is determined to be certain if a predetermined one or more of the plurality of features has been stable for a predetermined period of time, even if the risk score range is within the predetermined risk threshold.

According to embodiment, the method further includes: receiving, at the risk score analysis system, a second plurality of features about the patient, the second plurality of features comprising at least a plurality of vital signs obtained for the patient at a second time point subsequent to the first timepoint; updating, using the received second plurality of features, the initial risk score, the risk score confidence interval, and the risk score range; and presenting, to a user via a user interface of the risk score analysis system, the updated risk score range comprising both calculated initial risk scores and both calculated risk score ranges.

According to an embodiment, the risk score confidence interval comprises an effect of two or more missing features, and wherein the presented risk score range comprises an indication of the effect of each of the two or more missing features on the risk score range.

According to an embodiment, the presentation of the one or more of the identified one or more missing features comprises an identification of an importance of the respective missing feature to the risk score analysis.

According to an embodiment, the method further comprises training the trained risk model of the risk analysis system, comprising: receiving a training data set comprising a plurality of features obtained for a plurality of patients over a plurality of subsequent timepoints, each of the plurality of features for each of the plurality of patients comprising at least a plurality of vital signs obtained for the patient at each of the plurality of subsequent timepoints, and wherein the training data set comprises an outcome for each of the plurality of patients; processing the received training data set for training to generate a processed training dataset; and training, using the processed training dataset, the risk model of the risk analysis system to recognize an importance of a feature to a risk score at a given timepoint, and/or to recognize an effect of a feature on a confidence range of a risk score at a given time, to generate a trained risk model.

According to embodiment, an importance of a feature to a risk score at a given timepoint is based on a Shapley value of the feature at that timepoint.

According to another aspect is a patient risk score analysis system. The patient risk score analysis system comprises: a trained risk model configured to generate a risk score with confidence intervals from a plurality of received features about a patient, the plurality of received features comprising at least a plurality of vital signs obtained for the patient at a first time point; a processor configured to: (i) characterize, using the trained risk model, and importance of each of the received plurality of features at the first time point to a risk score analysis; (ii) calculate, from the received plurality of features about the patient, an initial risk score; (iii) identify, using the trained risk model, one or more missing features each comprising a feature not found in the plurality of received features, wherein each of the one or more missing features is relevant to the patient risk score calculation; (iv) calculate, using the trained risk model and the identified one or more missing features, a risk score confidence interval comprising an effect of the identified one or more missing features on a confidence range of the initial risk score; and (v) calculate, from the initial risk score and the calculated risk score confidence interval, a risk score range; and a user interface configured to present to a user the risk score range comprising the initial the score plus and minus the calculated risk score confidence interval, and one or more of the identified one or more missing features.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for generating and presenting a patient risk score, in accordance with an embodiment.

FIG. 2 is a schematic representation of a risk analysis system, in accordance with an embodiment.

FIG. 3 is a flowchart of a method for training a patient risk score model, in accordance with an embodiment.

FIG. 4 is a flowchart of a method for generating and presenting a patient risk score, in accordance with an embodiment.

FIG. 5 is a graph of a generated and presented risk score with a risk score confidence interval, in accordance with an embodiment.

FIG. 6 shows several graphs of individualized feature importance over time, in accordance with an embodiment.

FIG. 7 is a representation of an indication of flagged missing features, in accordance with an embodiment.

FIG. 8 is a graph showing decision trajectories for top features relevant to a risk score analysis at a current time, in accordance with an embodiment.

FIG. 9 is a graph showing a patient’s decision pathway mapped out against a group of low risk discharge patients, in accordance with an embodiment.

FIG. 10 is a graph showing a patient’s decision pathway mapped out against a group of high risk ICU-transfer patients, and accordance with an embodiment.

FIG. 11 shows graphs depicting algorithm performance metrics, in accordance with an embodiment.

### DETAILED DESCRIPTION OF EMBODIMENTS

The present disclosure describes various embodiments of the system and method configured to generate and present a patient risk score. More generally, Applicant has recognized and appreciated that it would be beneficial to provide a method and system to quantify and communicate uncertainty within a machine-learning patient risk score model. Accordingly, Applicant provides a risk score analysis system and method to quantify and communicate uncertainty in a patient with score model. The system receives a plurality of features about the patient, where the plurality of features comprise a plurality of vital signs obtained for the patient at a first time point. The system characterizes, using a trained risk model of the risk score analysis system, an importance of each of the received plurality of features at the first time point to a risk score analysis. The system calculates, from the received a plurality of features about the patient, an initial risk score. Then the system identifies, using the trained risk model, one or more missing features each comprising a feature not found in the plurality of received features, wherein each of the one or more missing features is relevant to the patient risk score calculation. The system calculates, using the trained risk model and the identified one or more missing features, a risk score confidence interval comprising an effect of the identified one or more missing features on a confidence range of the initial risk score, and then calculates, from the initial risk score and the calculated risk score confidence interval, a risk score range. The system prevents, to a user via a user interface of the risk score analysis system, the risk score range comprising the initial the score plus and minus the calculated risk score confidence interval, and one or more of the identified one or more missing features.

According to an embodiment, the systems and methods described or otherwise envisioned herein can, in some non-limiting embodiments, be implemented as an improvement to existing commercial products that incorporate disease staging and/or early warning scoring, such as an Intellivue Guardian bedside monitor, or a Central Station (both available from Koninklijke Philips NV, the Netherlands), or any suitable electronic health record system.

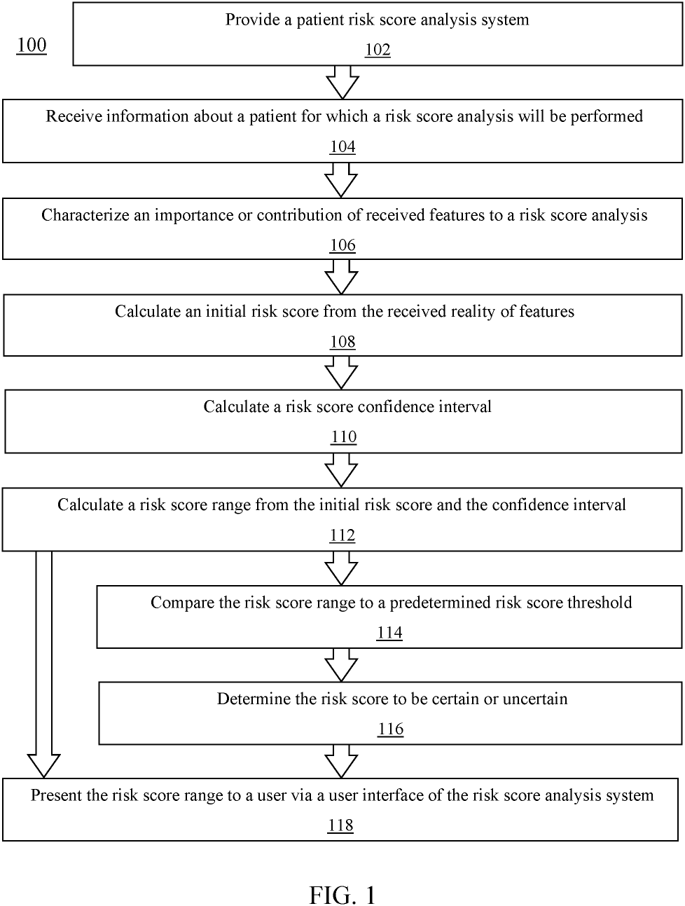

Referring to FIG. 1, in one embodiment is a flowchart of a method 100 for generating and communicating a patient risk score using a patient risk score analysis system. The methods described in connection with the figures are provided as examples only, and shall be understood not limit the scope of the disclosure. The patient risk score analysis system can be any of the system described otherwise envisioned herein. The patient risk score analysis system can be a single system or multiple different systems.

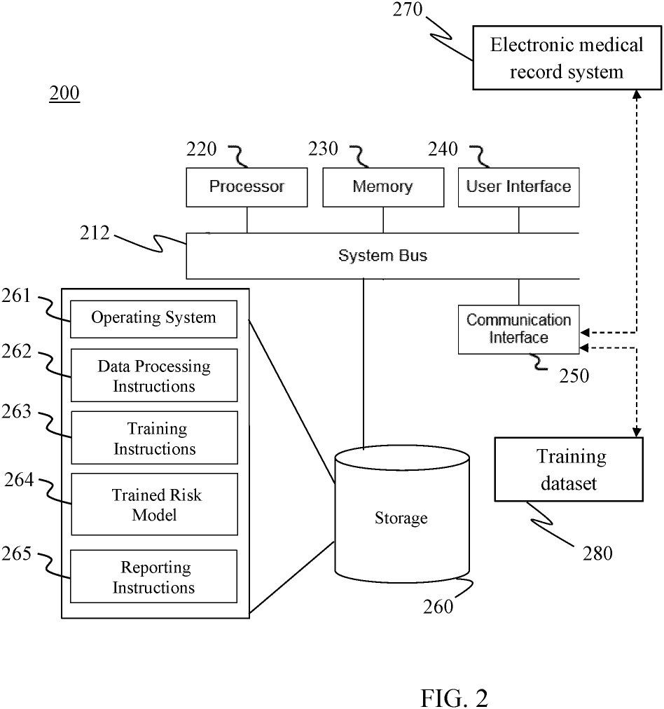

At step 102 of the method, a patient risk score analysis system is provided. Referring to an embodiment of a patient risk score analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, risk score analysis system 200 can be any of the systems described or otherwise envisioned herein. Other elements and components of risk score analysis system 200 are disclosed and/or envisioned elsewhere herein.

At step 104 of the method, the risk score analysis system receives information about a patient for which a risk score analysis will be performed. According to an embodiment, the information comprises a plurality of features about the patient. The plurality of features comprise vital sign information obtained for, about, or otherwise from the patient at a first timepoint. For example, the vital sign information may comprise physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. According to an embodiment, the information may also comprise medical information about the patient, including but not limited to demographics, physiological measurements such as vital data, physical observations, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more. Many other types of medical information are possible. Accordingly, the received information can be any information relevant to a patient risk score analysis.

According to an embodiment, the patient risk score analysis system may be in communication with an electronic medical records database from which the plurality of features may be obtained or received. The electronic medical records database may be a local or remote database and is in communication the patient risk score analysis system 200. According to an embodiment, the patient risk score analysis system comprises an electronic medical record database or system 270 which is optionally in direct and/or indirect communication with system 200. According to another embodiment, the patient risk score analysis system may obtain or receive the plurality of features from equipment or a healthcare professional obtaining that information directly from the patient.

At step 106 of the method, the system utilizes a trained risk model to characterize an importance or contribution of one or more of the received plurality of features to a risk score analysis at the first timepoint. Training of the trained patient risk model of the risk score analysis system is described elsewhere herein.

According to just one embodiment, the personalized feature importance (IFI) is based on Shapely Value, and is generated per the following:

$${ϕ}_{i} = {\sum }_{S ⊆ F \backslash \left\{i\right\}} \frac{\left|S\right| ! \left(\left|F\right| - \left|S\right| - 1\right) !}{\left|F\right| !} \left[{f}_{S ∪ \left\{i\right\}} \left({x}_{S ∪ \left\{i\right\}}\right) - {f}_{s} \left({x}_{s}\right)\right]$$

and the additive property:

$$g \left({z}^{′}\right) = {ϕ}_{0} + {\sum }_{i = 1}^{M} {ϕ}_{i} {z}_{i} {}^{′}$$

where ϕᵢ is the contribution of a given feature I — i.e. individualize feature importance; S is the coalition/all subsets of features used for the risk score model; F is the set of all features; f_{su{i}} is the model trained with that feature present; fₛ is the model trained with that feature withheld; xₛ is the value of the input features in the set D; ϕ₀ is the expected prediction of the model;

$${{z}^{′}}_{i}$$

∈ {0,1}^{M}; and M is the number f of input features. However other methods of generating the personalized feature importance for possible

At step 108 of the method, the patient risk score analysis system calculates, from the received plurality of features about the patient, and an initial risk score. The initial risk score may be calculated using any algorithm configured to analyze input such as patient data and generate a score or other similar analysis. Notably, the patient risk score analysis system may utilize any algorithm and is not model agnostic. Accordingly the system can utilize any algorithm that takes a set of numerical inputs and computes a or a set of numerical outputs. According to an embodiment, there are a wide variety of algorithms configured to analyze patient data and generate an initial risk score.

At step 110 of the method, the patient risk score analysis system identifies, using the trained risk model, one or more missing features each comprising a feature not found in the plurality of received features. According to an embodiment, each of the one or more missing features is relevant to the patient risk score calculation, and thus the missing feature affects the certainty or uncertainty of the patient risk score calculation or analysis. According to an embodiment, the number of identified missing features may depend upon the trained risk model and/or on a predetermined setting, perimeter, or threshold.

At step 112 of the method, the risk score analysis system calculates, using the trained risk model and the identified one or more missing features, a risk score confidence interval (SCI) comprising an effect of the identified one or more missing features on a confidence range of the initial risk score. According to an embodiment, the risk score confidence interval exploits the additive property by utilizing the IFI. For example, the standard deviation of IFI from all missing features can be summed to obtain the unit with of the confidence interval for the risk score. Thus, the uncertainty of the risk score can be apportioned to each individual input feature, for each patient, at each time point.

According to an embodiment the risk contributed by the imputed missing feature can be subtracted from the initial risk score. The missing feature variations can be added to compute the lower and upper limits of the confidence interval. According to an embodiment, a scaling factor can optimize the confidence interval lower and upper limits, such as by optimizing sensitivity and specificity, or through other optimization methodologies.

At step 112 of the method, the patient risk score analysis system calculates a risk score range using the initial risk score and the calculated risk score confidence interval. For example, the risk score range may comprise the initial risk score with a risk score confidence interval on either side of the risk score range. Embodiments of the risk score range comprising the initial risk score in the calculated risk score confidence interval are described elsewhere herein.

At step 118 of the method, the patient risk score analysis system presents to a user via a user interface the risk score range comprising the initial the score plus and minus the calculated risk score confidence interval. According to an embodiment, the system also presents to the user via the user interface one or more of the identified one or more missing features.

According to an embodiment, the method returns to step 104 to receive a second plurality of features about the patient, the second plurality of features comprising at least a plurality of vital signs obtained for the patient at a second time point subsequent to the first timepoint. As described elsewhere herein, the plurality of features may comprise a wide variety of different information about the patient.

The method repeat steps 106, 108, 110, and 112 to update, using the received second plurality of features, the initial risk score, the risk score confidence interval, and the risk score range. Then, at step 118 of the method, the system presents to the user via the user interface, the updated risk score range comprising both calculated initial risk scores and both calculated risk score ranges.

At optional step 114 of the method, the patient risk score analysis system compares the risk score range to a predetermined risk score threshold. The predetermined risk score threshold may be determined by the trained risk model, by a user, or by other parameters or settings within the risk score analysis system.

At optional step 116 of the method, the trained risk model of the patient risk score analysis system determines the risk score to be certain if the risk score range is outside the predetermined risk score threshold. According to an embodiment, the trained risk model of the patient risk score analysis system determines the risk score to be uncertain if the risk score range is within the predetermined risk score threshold.

According to an embodiment, if the confidence interval is outside the risk score threshold, then the system determines the prediction to be uncertain. Otherwise, the system may determine the risk score to be certain. According to an embodiment, the system may abstain is classification prediction for all cases deemed to be uncertain. This approach may be the most stringent for flagging uncertain cases, and thus can be designed to encourage physician interpretation as much as possible.

According to another embodiment, if the risk score has been stable for a pre-defined timeframe in the past, and the confidence interval is within a pre-defined or otherwise predetermined range, the system can determine the risk score to be certain despite the fact that it may contain the risk score threshold.

According to another embodiment, if the risk score and one or more top contributing features have been stable for a pre-defined or otherwise predetermined timeframe in the past, and the confidence interval is within a pre-defined or otherwise predetermined range, the system can determine the risk or to be certain despite the fact that it may contain the risk score threshold.

According to another embodiment, the risk score analysis system may assign different risk score intervals for each feature in the risk score confidence interval calculation based on the value of the risk score. For example, intervals can be assigned to bins such as above the risk or threshold, below the risk score threshold, and other business.

According to an embodiment, if an input feature is stable for a pre-defined or otherwise predetermined timeframe in the past, its value can be carried over more to adjust its base-risk in the risk score and eliminate its contribution to the confidence interval.

Returning to step 118 of the method, the system presents to the user via the user interface, the determination of the risk score to be certain or uncertain.

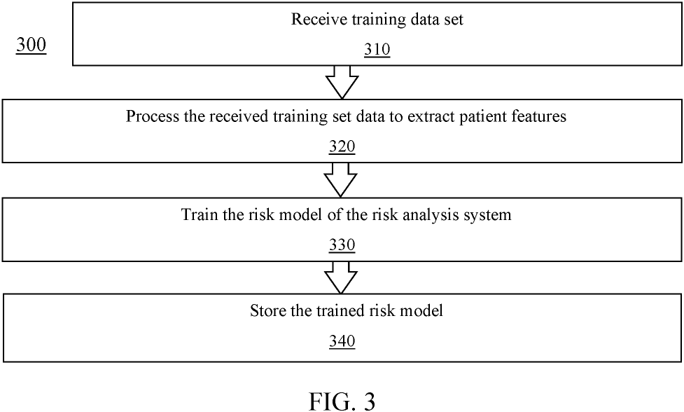

Referring to FIG. 3, in one embodiment, is a flowchart of a method 300 for training the risk model of the risk analysis system. At step 310 of the method, the system receives a training data set comprising training data about a plurality of patients. The training data can comprise medical information about each of the patients, including but not limited to demographics, physiological measurements such as vital data, physical observations, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types of medical information are possible. According to an embodiment, the training data may also comprise an indication or information about one or more outcomes of each patient.

This training data may be stored in and/or received from one or more databases. The database may be a local and/or remote database. For example, the patient risk analysis system may comprise a database of training data.

According to an embodiment, the patient risk analysis system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the system extracts patient features from the received training data. This can be accomplished by a variety of embodiments for feature identification, extraction, and/or processing, including any method for extracting features from a dataset. The outcome of a feature processing step or module of the patient risk analysis system is a set of patient features related to medical information and clinical states about a patient, which thus comprises a training data set that can be utilized to train the classifier.

At step 330 of the method, the system trains the machine the learning algorithm, which will be the algorithm utilized in analyzing patient information as described or otherwise envisioned. The machine learning algorithm is trained using the extracted features according to known methods for training a machine learning algorithm. According to an embodiment, the algorithm is trained, using the processed training dataset, to recognize an importance of a feature to a risk score at a given timepoint, and/or to recognize an effect of a feature on a confidence range of a risk score at a given time, to generate a trained risk model.

Following step 330 of the method, the risk analysis system comprises a trade algorithm or model or classifier that can be utilized to generate a risk analysis as described or otherwise envisioned. The trained classifier can be static such that it is trained once and is utilized for classifying. According to another embodiment, the trained classifier can be more dynamic such that it is updated or re-trained using subsequently available training data. The updating or re-training can be constant or can be periodic.

At step 340 of the method, the trained algorithm can be stored locally or remotely for subsequent analysis of patient features.

Referring to FIG. 2 is a schematic representation of a patient risk analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, an electronic medical record system 270, training dataset 280, data processing instructions 262, training instructions 263, trained risk model 264, and/or reporting instructions 265.

According to an embodiment, the electronic medical record system 270 is an electronic medical records database from which the plurality of features may be obtained or received. The electronic medical records database may be a local or remote database and is in communication the patient risk score analysis system 200. According to an embodiment, the patient risk score analysis system comprises an electronic medical record database or system 270 which is optionally in direct and/or indirect communication with system 200.

According to an embodiment, the training data set 280 is a dataset may be stored in a database that may be a local or remote database and is in communication the patient risk score analysis system 200. According to an embodiment, the patient risk score analysis system comprises a training data set 280. The training data can comprise medical information about each of the patients, including but not limited to demographics, physiological measurements such as vital data, physical observations, and/or diagnosis, among many other types of medical information. According to an embodiment, the training data may also comprise an indication or information about one or more outcomes of each patient.

According to an embodiment, data processing instructions 262 direct the system to retrieve and process input data which is used to train the risk model 264. The data processing instructions 262 direct the system to for example, receive or retrieve input data or medical data to be used by the system as needed, such as from electronic medical record system 270 among many other possible sources. As described above, the input data can comprise a wide variety of input types from a wide variety of sources.

According to an embodiment, the data processing instructions 262 also direct the system to process the input data to generate a plurality of features related to medical information for a plurality of patients, which are used to train the classifier. This can be accomplished by a variety of embodiments for feature identification, extraction, and/or processing. The outcome of the feature processing is a set of features related to risk analysis for a patient, which thus comprises a training data set that can be utilized to train the risk model 264.

According to an embodiment, training instructions 263 direct the system to utilize the process data to train the risk model 264. The risk model can be any machine learning algorithm, classifier, or model sufficient to utilize the type of input data provided, and to generate a risk analysis. Thus, the system comprises a trained risk model 264 configured to generate a risk prediction or a patient.

According to an embodiment reporting instructions 265 direct the system to generate and provide a report to a user via a user interface comprising a generated risk score range. According to an embodiment, the risk score range comprises the initial the score with an indication of the calculated risk score confidence interval. According to an embodiment, the system also presents to the user via the user interface one or more of the identified one or more missing features.

According to an embodiment, the reporting instructions 267 direct the system to display the report on a display of the system. The display may comprise information about the patient, the parameters, the input data for the patient, and/or the patient’s risk. Other information is possible. Alternatively, the report may be communicated by wired and/or wireless communication to another device. For example, the system may communicate the report to a mobile phone, computer, laptop, wearable device, and/or any other device configured to allow display and/or other communication of the report.

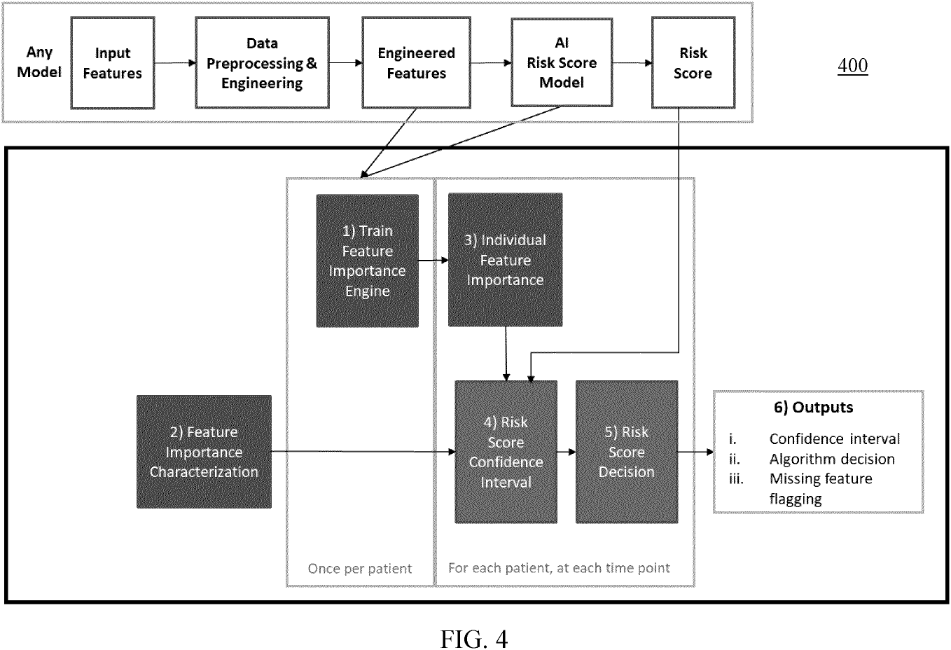

Referring to FIG. 4, in one embodiment, is a flowchart of a method 400 for generating and communicating a patient risk score using a patient risk score analysis system. The method described in connection with this figure is provided as an example only, and shall be understood not limit the scope of the disclosure. The patient risk score analysis system can be any of the system described otherwise envisioned herein.

According to an embodiment, a risk score model (“AI Risk Score Model”) is trained using a training dataset (“Input Features”) as described or otherwise envisioned herein. The input data is preprocessed and engineered as described or otherwise envisioned herein (“Data Preprocessing & Engineering”) to function as the training dataset for the risk score model. The resulting training dataset (“Engineered Features”) are utilized to train the risk score model (“AI Risk Score Model”), which can be utilized to generate a Risk Score. The risk score model can comprise, for example, a Feature Importance Engine that is configured to identify the importance or contribution of patient features to a risk score analysis. With a trained model, the risk score analysis system can be utilized to generate risk scores and confidence intervals for individual patients. Accordingly, upon receiving patient features for a patient, the risk score model determines the importance of one or more of the received patient features at the first time point to the risk score analysis. The model can also identify one or more missing features each comprising a feature not found in the plurality of received features. Then, the system can calculate, from the initial risk score and the identified one or more missing features, a Risk Score Confidence Interval. Optionally, this can be utilized to determine a Risk Score Decision as described or otherwise envisioned herein.

The system provides an output to the user comprising at least the risk score and the confidence interval for that risk score. The system may also provide the Risk Score Decision (“Algorithm decision”) and/or an identification of one or more of the identified missing features (“Missing feature flagging”).

### Example 1

Discussed below are two examples of how the patient risk analysis system might be utilized. It should be appreciated that these examples are non-limiting. For example, patient risk scores can be provided for a wide variety of different conditions, events, outcomes, or other aspects of patient care. Discussed below is an application of the methods and systems described or otherwise envisioned herein for acute heart failure patients. As applied to acute heart failure patients, the individualized risk score interpretation provided by the methods and systems described herein aid clinical decisions and transitions of care.

Acute heart failure is a complex disease with heterogenous manifestations. The interpretation of machine-learning risk scores is vital for their ability to support clinical decisions and transitions of care. Individualized Feature Importance (IFI) was designed and applied to attribute changes in risk scores to clinical features and help contrast decision trajectory for a patient against those of patient subgroups that received distinct clinical decisions. Score Confidence Interval (SCI) was developed and applied to quantify the level of certainty in the prediction, which reduces false alarm rates and further encourages clinicians’ interpretation.

This study was based on retrospective data from 25 hospitals in the U.S. comprising 20,640 adult patients between 2014 to 2018, with 87% discharged home (Class 0) and 13% transferred to the ICU or died in hospital (Class 1). In this analysis, IFI is based on Shapley Value, based on which SCI was designed to capture the variation in score if input features are missing. These methods were applied to previously developed risk score for AHF patients in the wards; however, they can be applied to any risk score.

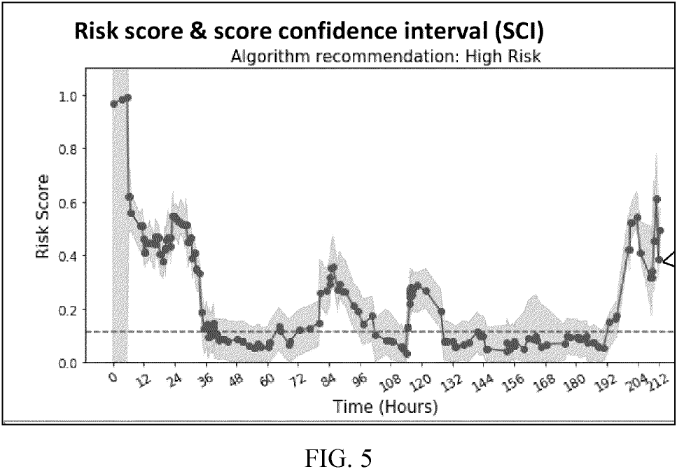

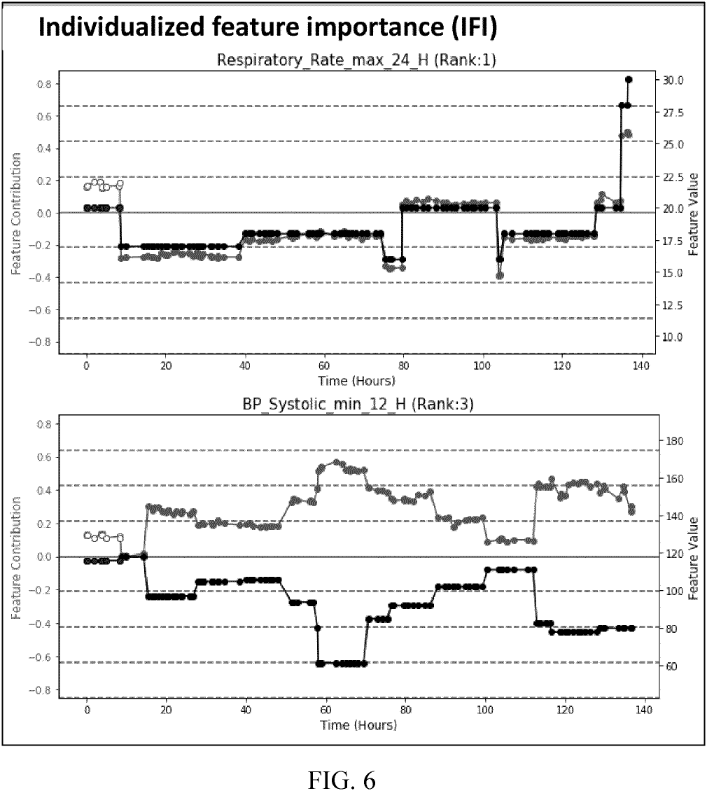

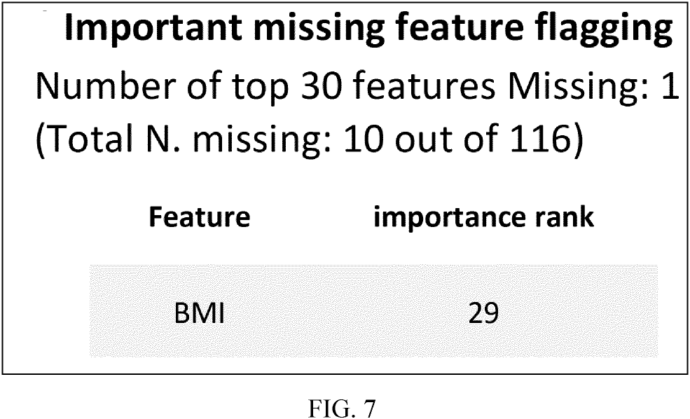

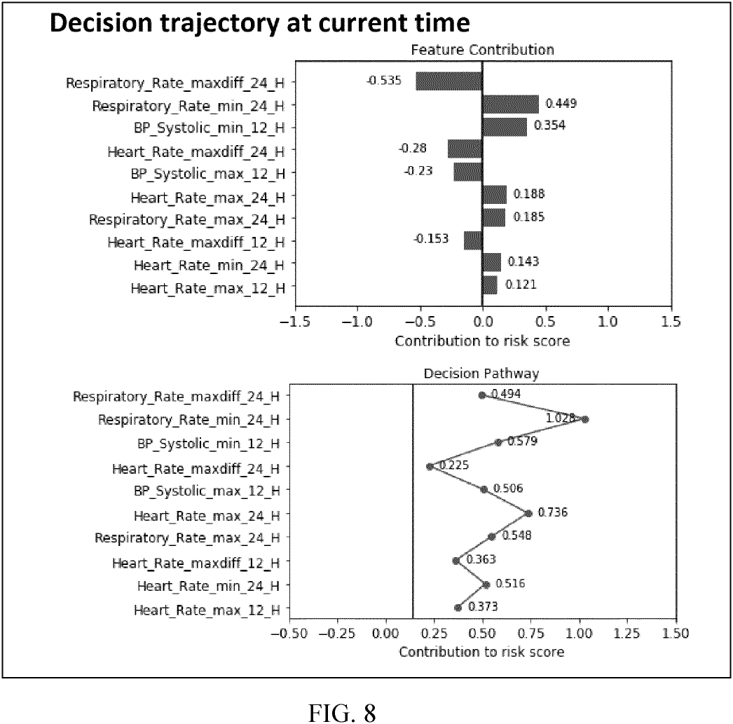

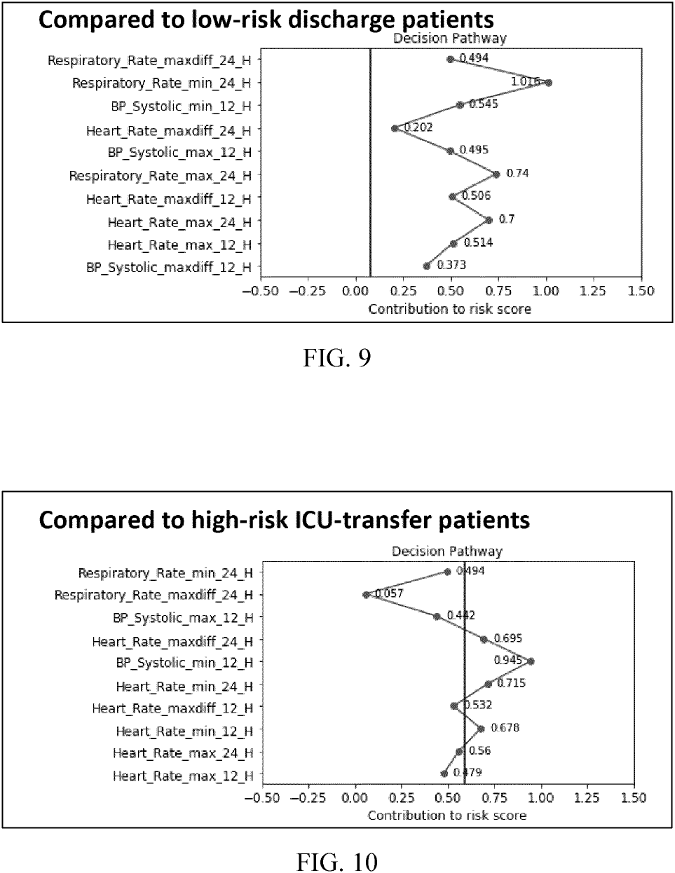

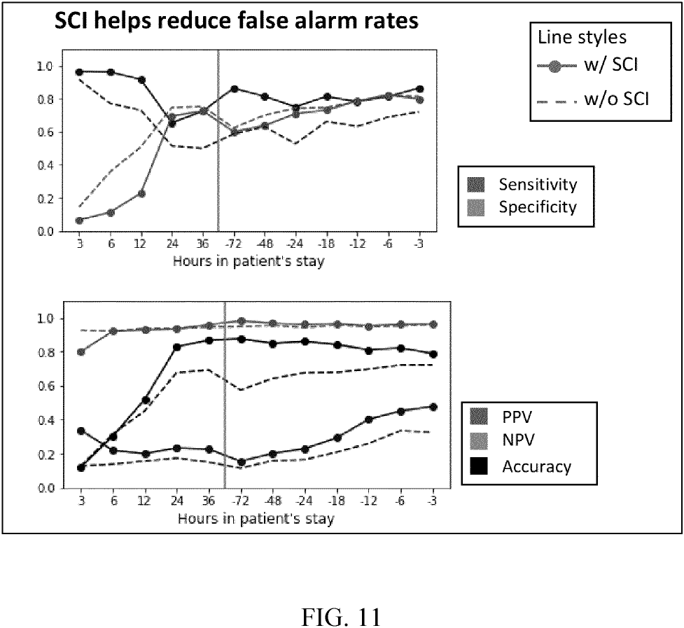

Referring to FIGS. 5-11, the SCI was wide at the beginning of the stay and narrowed down towards the end as more clinical measurements becomes available, indicating the risk score is relatively certain at the end, as shown in FIG. 5. Referring to FIG. 6, IFI values show how selected features drive changes in the risk score. To aid decision-making the at the latest time, top missing features are prompted, as shown in FIG. 7. Decision trajectories show the way top features drive the risk score, as shown in FIG. 8, and that this patient is at higher risk to discharges, as shown in FIG. 9, and is more similar to ICU-transfers as shown in FIG. 10. FIG. 11 shows SCI improves risk score performance by abstaining uncertain cases from decision-making. Accordingly, IFI apportions risk score to clinical measurements, and SCI reduces false alarm rates. By providing clinical context, they have the potential to enhance incorporation of telemedicine in the clinical workflow.

Referring to FIG. 4, the risk score (line) reflects extent of deterioration of acute heart failure patients and predicts discharge (below threshold indicated by dash line) or escalation to the ICU (above threshold). It is updated (dot) whenever a new input clinical feature becomes available. The SCI is overlaid on the risk score (lighter regions). According to an embodiment, if the SCI region contains the threshold, the score is deemed uncertain.

Referring to FIG. 6 are IFI shown across time for selected input features. The ranking for each feature is provided in brackets in the title according to IFI for the patient at the latest time point, with more important features receiving a higher rank. The left-hand side axis (gray) indicates IFI values (gray line), which when summed up across all input features equals the risk score. The right-hand side axis (black) show actual feature values (black line). Horizontal dashed blue lines show standard deviations of IFI values for the given feature on pre-selected training set. Some feature values are imputed when the actual value is missing, and the corresponding IFI value is filled. Selected features are plotted (titles): Respiratory_Rate_max_24H: maximum respiratory rate in the past 24 hours; BP_systolic_min_12_H: minimum systolic blood pressure in the past 12 hours. Other possible features include: SI_mean_12_H: Mean of Shock Index (SI) in the past 12 hours; Bun_Creat_Ratio: BUN-creatinine ratio; and Fluid Balance_24_H: fluid balance in the past 24 hours.

Referring to FIG. 7, missing features with high IFI at the latest time point are identified.

Referring to FIG. 8, for the last time point, a decision trajectory shows how input features sum up to the score in FIG. 5. Features are ranked by their IFI values for the given patient at the latest time point, with the most important appearing first on top. The top ten contributing features are shown. The vertical lines indicate the expected risk scores for respective patient subgroups derived from pre-selected training sets. The patient’s decision pathway is mapped out against a group representative of all AHF patients. The top panel shows the values that the selected features contributed to the risk score, and the bottom panel shows an alternative visualization to the decision trajectory.

Referring to FIG. 9, the patient’s decision pathway is mapped out against a group of low-risk discharge patients for comparison. Referring to FIG. 10, the patient’s decision pathway is mapped out against a group of high-risk ICU-transfer patients for comparison.

Referring to FIG. 11, in one embodiment, algorithm performance metrics with SCI (solid line) and without SCI (dashed line) are shown for selected hours from admission (positive hours) and prior to disposition (negative hours). Cases deemed uncertain by SCI are abstained from decision-making. SCI was evaluated on randomly-selected 20% of unseen test set with prevalence of classes preserved. The top panel shows sensitivity and specificity, and the bottom panel shows Positive Predictive Value (PPV), Negative Predictive Value (NPV), and Accuracy.

In addition to acute heart failure, the methods, systems, and devices disclosed or otherwise envisioned herein can be utilized for a wide variety of other patient scores, analyses, conditions, or other situations. Any analysis that utilizes a risk score analysis, or could benefit from a risk score, could be a component or focus of this system.

As just one example, the methods, systems, and devices disclosed or otherwise envisioned herein can be utilized for a risk score analysis related to early deterioration index, in which general patient deterioration is detected and the system can predict when a patient should be transferred to a unit, location, or treatment with a more intensive level of care.

As yet another example, the methods, systems, and devices disclosed or otherwise envisioned herein can be utilized for a risk score analysis related to hemodynamics stability index, in which the system can predict when a patient will need medication or other interventions for hemodynamic instability such as shock.

These examples should be noted as just possible examples of the methods, systems, and devices disclosed or otherwise envisioned herein, and are thus non-limiting examples.

According to an embodiment, the patient risk score analysis system is configured to process many thousands or millions of datapoints in the input data used to train the classifier, as well as to process and analyze the received plurality of patient features. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm, and thus improves the functioning of the risk score analysis system. Thus, generating a functional and skilled trained classifier comprises a process with a volume of calculation and analysis that a human brain cannot accomplish in a lifetime, or multiple lifetimes.

In addition, the patient risk score analysis system can be configured to continually receive patient features, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime.

By providing an improved patient risk score analysis, this novel patient risk score analysis system has an enormous positive effect on patient risk analysis compared to prior art systems. As just one example in a clinical setting, by providing a system that can improve patient risk scores with confidence intervals, the system can facilitate treatment decisions and improve survival outcomes, thereby leading to saved lives.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for generating and presenting a patient risk score using risk score analysis system, comprising:
receiving, at the risk score analysis system, a plurality of features about the patient, the plurality of features comprising at least a plurality of vital signs obtained for the patient at a first time point;
characterizing, using a trained risk model of the risk score analysis system, an importance of each of the received plurality of features at the first time point to a risk score analysis;
calculating, from the received plurality of features about the patient, an initial risk score;
identifying, using the trained risk model, one or more missing features each comprising a feature not found in the plurality of received features, wherein each of the one or more missing features is relevant to the patient risk score calculation;
calculating, using the trained risk model and the identified one or more missing features, a risk score confidence interval comprising an effect of the identified one or more missing features on a confidence range of the initial risk score;
calculating, from the initial risk score and the calculated risk score confidence interval, a risk score range; and
presenting, to a user via a user interface of the risk score analysis system, the risk score range comprising the initial the score plus and minus the calculated risk score confidence interval, and one or more of the identified one or more missing features.

2. The method of claim 1, further comprising:
comparing the risk score range to a predetermined risk score threshold;
determining, by the trained risk model, the risk score to be certain if the risk score range is outside the predetermined risk score threshold, or determining the risk score to be uncertain if the risk score range is within the predetermined risk score threshold; and
presenting, to the user via the user interface, the determination of the risk score to be certain or uncertain.

3. The method of claim 2, wherein the risk score is determined to be certain if the risk score has been stable for a predetermined period of time, even if the risk score range is within the predetermined risk score threshold.

4. The method of claim 2, wherein the risk score is determined to be certain if a predetermined one or more of the plurality of features has been stable for a predetermined period of time, even if the risk score range is within the predetermined risk threshold.

5. The method of claim 1, further comprising the steps of:
receiving, at the risk score analysis system, a second plurality of features about the patient, the second plurality of features comprising at least a plurality of vital signs obtained for the patient at a second time point subsequent to the first timepoint;
updating, using the received second plurality of features, the initial risk score, the risk score confidence interval, and the risk score range; and
presenting, to a user via a user interface of the risk score analysis system, the updated risk score range comprising both calculated initial risk scores and both calculated risk score ranges.

6. The method of claim 1, wherein the risk score confidence interval comprises an effect of two or more missing features, and wherein the presented risk score range comprises an indication of the effect of each of the two or more missing features on the risk score range.

7. The method of claim 1, wherein the presentation of the one or more of the identified one or more missing features comprises an identification of an importance of the respective missing feature to the risk score analysis.

8. The method of claim 1, further comprising training the trained risk model of the risk analysis system, comprising:
receiving a training data set comprising a plurality of features obtained for a plurality of patients over a plurality of subsequent timepoints, each of the plurality of features for each of the plurality of patients comprising at least a plurality of vital signs obtained for the patient at each of the plurality of subsequent timepoints, and wherein the training data set comprises an outcome for each of the plurality of patients;
processing the received training data set for training to generate a processed training dataset; and
training, using the processed training dataset, the risk model of the risk analysis system to recognize an importance of a feature to a risk score at a given timepoint, and/or to recognize an effect of a feature on a confidence range of a risk score at a given time, to generate a trained risk model.

9. The method of claim 8, wherein an importance of a feature to a risk score at a given timepoint is based on a Shapley value of the feature at that timepoint.

10. A patient risk score analysis system, comprising:
a trained risk model configured to generate a risk score with confidence intervals from a plurality of received features about a patient, the plurality of received features comprising at least a plurality of vital signs obtained for the patient at a first time point;
a processor configured to: (i) characterize, using the trained risk model, and importance of each of the received plurality of features at the first time point to a risk score analysis; (ii) calculate, from the received plurality of features about the patient, an initial risk score; (iii) identify, using the trained risk model, one or more missing features each comprising a feature not found in the plurality of received features, wherein each of the one or more missing features is relevant to the patient risk score calculation; (iv) calculate, using the trained risk model and the identified one or more missing features, a risk score confidence interval comprising an effect of the identified one or more missing features on a confidence range of the initial risk score; and (v) calculate, from the initial risk score and the calculated risk score confidence interval, a risk score range;
a user interface configured to present to a user the risk score range comprising the initial the score plus and minus the calculated risk score confidence interval, and one or more of the identified one or more missing features.

11. The system of claim 10, wherein the processor is further configured to compare the risk score range to a predetermined risk score threshold; and further wherein the trained risk model is configured to determine the risk score to be certain if the risk score range is outside the predetermined risk score threshold, and to determine the risk score to be uncertain if the risk score range is within the predetermined risk score threshold; and wherein the user interface is further configured to present the determination of the risk score to be certain or uncertain.

12. The system of claim 11, wherein the risk score is determined to be certain if the risk score has been stable for a predetermined period of time, even if the risk score range is within the predetermined risk score threshold.

13. The system of claim 11, wherein the risk score is determined to be certain if a predetermined one or more of the plurality of features has been stable for a predetermined period of time, even if the risk score range is within the predetermined risk threshold.

14. The system of claim 10, wherein the processor is further configured to: receive a second plurality of features about the patient, the second plurality of features comprising at least a plurality of vital signs obtained for the patient at a second time point subsequent to the first timepoint; and update the initial risk score, the risk score confidence interval, and the risk score range using the received second plurality of features; and wherein the user interface is further configured to present the updated risk score range comprising both calculated initial risk scores and both calculated risk score ranges.

15. The system of claim 10, wherein the risk score confidence interval comprises an effect of two or more missing features, and wherein the presented risk score range comprises an indication of the effect of each of the two or more missing features on the risk score range.
